(19) United States
(12) Patent Application Publication
(10) Pub. No.: US 2026/0026739 A1
(43) Pub. Date: Jan. 29, 2026

(54) DEVICE FOR TEMPOROMANDIBULAR JOINT DISORDER DIAGNOSIS AND TREATMENT

(71) Applicant: Ethan Andrew KIBE, Louisville, CO (US)

(72) Inventor: Ethan Andrew KIBE, Louisville, CO (US)

(21) Appl. No.: 18/783,240

(22) Filed: Jul. 24, 2024

(51) Int. Cl. A61B 5/00; A61B 90/00

(52) U.S. Cl. CPC A61B 5/4542; A61B 5/4836; A61B 5/682; A61B 5/6838; A61B 2090/3945

(57) ABSTRACT

A device for diagnosing and treating temporomandibular joint (TMJ) disorder (TMD) in patients is disclosed. It includes a chin rest, a stem, a housing for a pointing device, and an oral clasp. The chin rest, which can be shaped like a quarter-sphere, is connected to the stem, which can be adjustable along its longitudinal axis. The housing, attached to the chin rest, holds a pointing device that projects a pointer on an observation plane in front of the patient. The oral clasp, which can be a dental clasp or mouth guard, is insertable into the patient's mouth and attached to the stem. The pointing device, which can be a laser or writing instrument, may be permanently attached or detachable. The device can be made of plastic materials such as polylactic acid (PLA) or acrylonitrile butadiene styrene (ABS).

### BACKGROUND

Temporomandibular joint (TMJ) disorder is a condition that causes pain, discomfort, and tenderness in your jaw joints and surrounding muscles and ligaments. This condition can cause several issues, including jaw and muscle pain, headaches, and difficulty opening and closing of the mouth. The exact cause of a person's TMJ disorder is often difficult to determine. Causes may be due to a combination of factors, such as genetics, stress, jaw injuries, teeth grinding, arthritis, and everyday wear and tear.

In anatomy, the TMJs are joints connecting the jawbone to the skull. People have two TMJs, one in front of each ear, that act like sliding hinges, which help with movements like chewing and speaking. It is a bilateral synovial articulation between the temporal bone of the skull above and the lower jawbone (mandible) below. This joint is unique in that it is a bilateral joint that functions as one unit. Since the TMJ is connected to the mandible, the right and left joints must function together and therefore are not independent of each other.

In most cases, the pain and discomfort associated with TMJ disorder (TMD) is temporary and can be relieved with self-managed care or nonsurgical treatments. TMD treatment varies from person to person and may include medication, physical therapy, custom mouth guards and jaw surgery. Surgery is typically a last resort after conservative measures have failed, but some people with TMD may benefit from surgical treatments. Between 5% and 12% of the general adult population have some form of TMD.

### BRIEF DESCRIPTION OF THE DRAWINGS

FIG. 1A is a front isometric view of an oral device for TMD diagnosis and treatment.

FIG. 1B is a back isometric view of the oral device.

FIG. 2A is a right isometric view of the oral device.

FIG. 2B is a right isometric view of an embodiment of the oral device with a laser-pointer device coupled with the housing.

FIG. 3 is an anterior elevation view of the oral device partially placed within the mouth of a patient.

FIG. 4 is a buccal elevation view of the oral device partially placed within the mouth of the patient.

FIG. 5A is a buccal elevation view of the oral device partially placed within the mouth of a patient, illustrating the device's positioning relative to the patient's teeth.

FIG. 5B is a buccal elevation view of an embodiment of the oral device with a laser-pointer device coupled with the housing and with the oral device partially placed within the mouth of a patient, illustrating the device's positioning relative to the patient's teeth.

FIG. 6 is a flowchart of a method for diagnosing and treating TMD in the patient.

### DETAILED DESCRIPTION

The disclosed technology pertains to a device for diagnosing, and treating, a TMD in a patient. The device comprises a chin rest, a superior end of which is coupled to a stem that terminates in an oral clasp and an inferior end of which is coupled to a pointer housing configured to hold a pointing device. The pointer housing can be referenced herein as the housing. In some embodiments, the pointing device can be a laser. The patient who is to be assessed for TMD can wear the device by resting her chin on the chin rest and placing the oral clasp inside her mouth. In some embodiments, the patient can open and close her lower jaw, thereby moving the device along with it and causing a pointer of the pointing device to trace a path on an observation plane in front of the patient. The path traced by the pointer can have a varying degree of irregularity depending on whether the patient suffers from severe, mild, or no TMD. The patient can be diagnosed for TMD, or a lack thereof, by determining whether the path traced by the pointer exceeds a threshold of at least one measure of irregularity. Further, a patient who has been diagnosed with TMD can use the device to exercise and train her TMJ by repeatedly opening and closing her jaw while using the device, observing the pattern traced by the pointer on the observation plane and adjusting the position and movement of her TMJ to reduce the degree of irregularity of the traced path.

The description and associated drawings are illustrative examples and are not to be construed as limiting. This disclosure provides certain details for a thorough understanding and enabling description of these examples. One skilled in the relevant technology will understand, however, that the invention can be practiced without many of these details. Likewise, one skilled in the relevant technology will understand that the invention can include well-known structures or features that are not shown or described in detail to avoid unnecessarily obscuring the descriptions of examples.

### Semi-Oral Device for Diagnosing and Treating a Face/Jaw Disorder

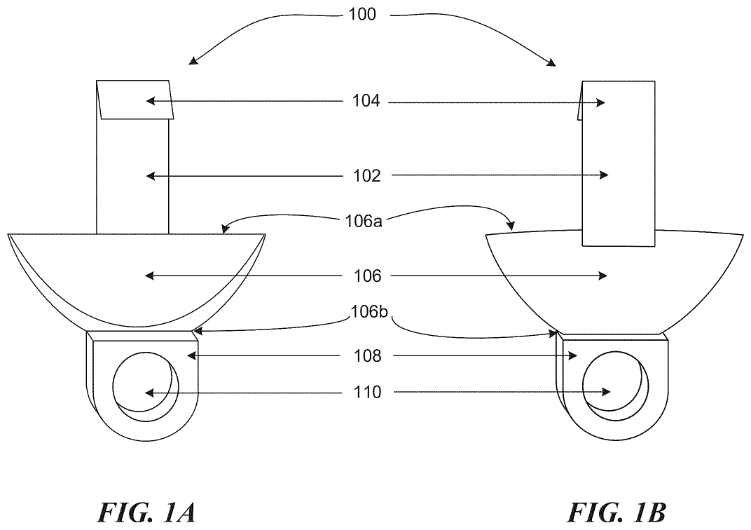

FIG. 1A is a front isometric view of a semi-oral device 100 (also referred to herein as an “oral device” or a “device”). FIG. 1B is a back isometric view of the oral device 100. The device comprises a chin rest 106, a stem 102, an oral clasp 104, and a housing 108. In some embodiments, the oral clasp 104 can be a dental clasp that can be worn by the patient over her lower front teeth. In some embodiments, the chin rest 106 can comprise a first cavity formed by a hollow quarter-sphere. The chin rest 106 can include a superior end 106a and an inferior end 106b. The housing 108 can include a second cavity 110 whereby the pointing device is coupled to the housing 108. In some embodiments, the second cavity 110 can have an annular shape. In some embodiments, the pointing device can be permanently coupled with the device 100. In some embodiments, the pointing device can be separated from the housing 108 when not in use.

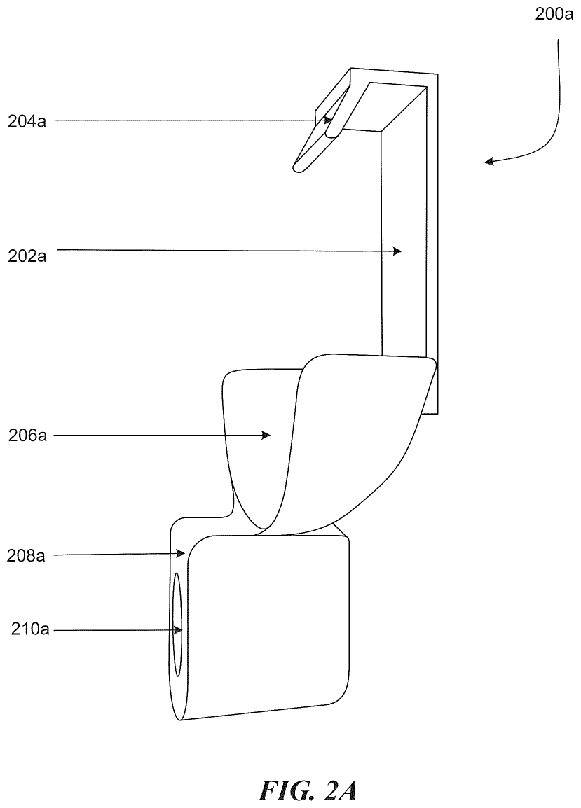

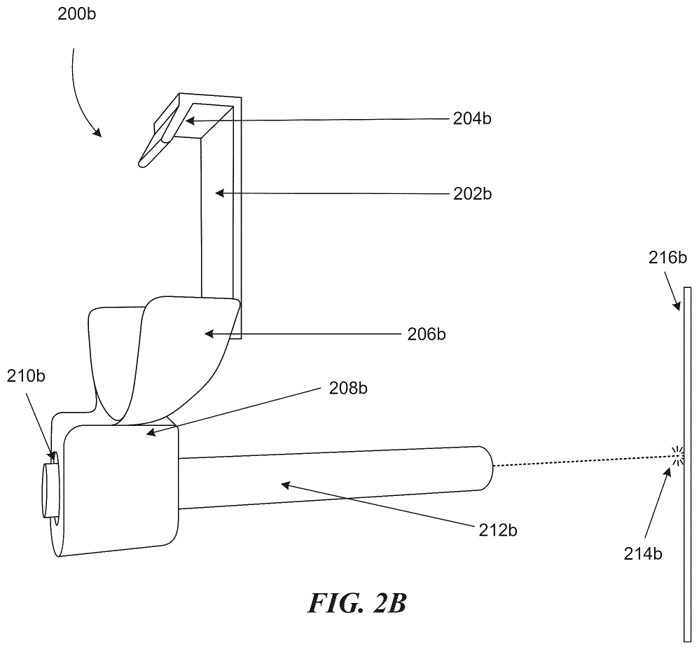

FIG. 2A is a right isometric view of the device 200a. The device 200a comprises a chin rest 206a, a stem 202a, an oral clasp 204a, and a housing 208a. In some embodiments, the chin rest 206a can comprise a first cavity formed by a hollow quarter-sphere. The housing 208a can include a second cavity 210a whereby the pointing device is coupled to the housing 208a. In some embodiments, the second cavity 210a can have an annular shape. In some embodiments, the pointing device can be permanently coupled with the device 200a. In some embodiments, the pointing device can be separated from the housing 208a when not in use.

FIG. 2B is a right isometric view of an embodiment of the device 200b with a pointing device 212b coupled with the housing 208b. In some embodiments, the housing 208b includes a cavity 210b at which the pointing device 212b is coupled to the housing 208b. The device 200b comprises a chin rest 206b, a stem 202b, an oral clasp 204b, and a housing 208b. In some embodiments, the pointing device 212b can be an optical pointing device. In some embodiments, the optical pointing device can be a laser. In some embodiments, the pointing device 212b can be a writing instrument such as a pen or a pencil. A pointer 214b of the pointing device 212b can be observed in an observation plane in front of the device 200b. In some embodiments, the observation plane can include an observation surface 216b. In some embodiments, the observation surface can be positioned at a configurable distance in front of the patient. For a patient not suffering from TMD, the pointer 214b can trace a regular path on the observation plane. For a patient suffering from TMD, the pointer 214b can trace an irregular path on the observation plane based on the extent of the patient's disorder. In some embodiments, for a patient suffering from mild TMD, the path traced by the pointer 214b on the observation plane can exceed a first threshold of a measure of irregularity. In some embodiments, for a patient suffering from severe TMD, the path traced by the pointer 214b on the observation plane can exceed a second threshold of a measure of irregularity. In some embodiments, for a patient not suffering from TMD, the path traced by the pointer 214b on the observation plane does not exceed the first or the second threshold of the measure of irregularity. In some embodiments, the measure of irregularity can be an angle made by the path traced by the pointer 214b on the observation plane with reference to a vertical plane passing perpendicularly through the observation plane. In some embodiments, the measure of irregularity can be a width of an aggregate path traced by the pointer 214b on the observation plane after a plurality of motions of the TMJ by the patient. In some embodiments, the observation surface 216b can include reference markings to calculate a measure of irregularity of the path traced by the pointer 214b on the observation surface 216b.

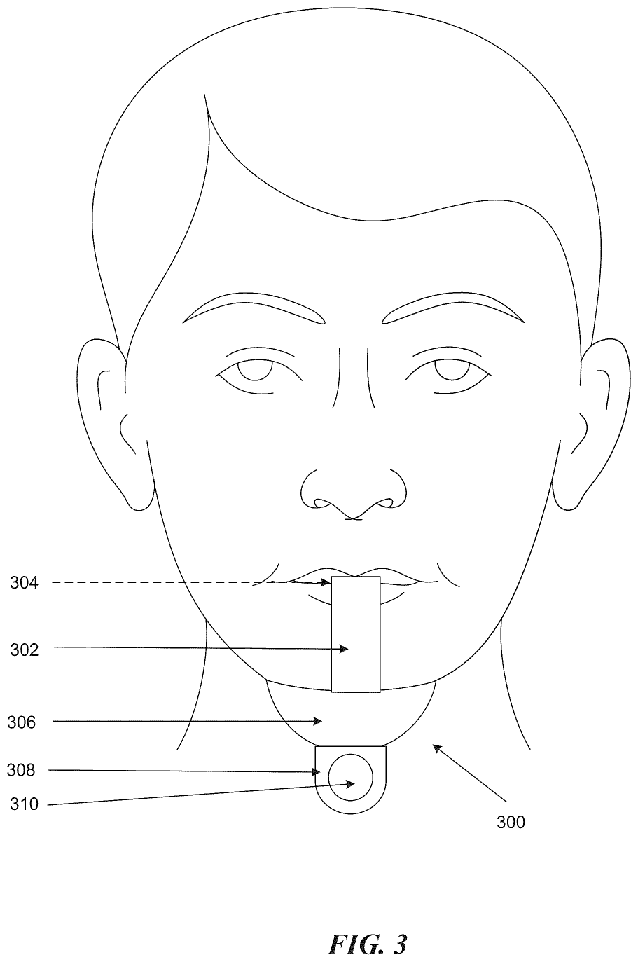

FIG. 3 is an anterior elevation view of the oral device 300 partially placed within the mouth of the patient. The structures and functions of the chin rest 306, the stem 302, the oral clasp 304, the housing 308, and the second cavity 310 are as described in preceding paragraphs.

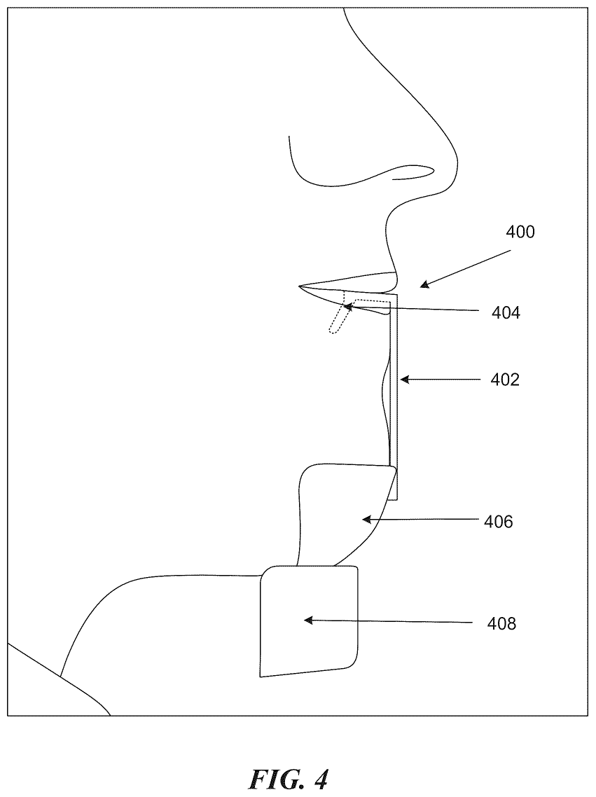

FIG. 4 is a buccal elevation view of the oral device 400 partially placed within the mouth of the patient. The structures and functions of the chin rest 406, the stem 402, the oral clasp 404, and the housing 408 are as described in preceding paragraphs.

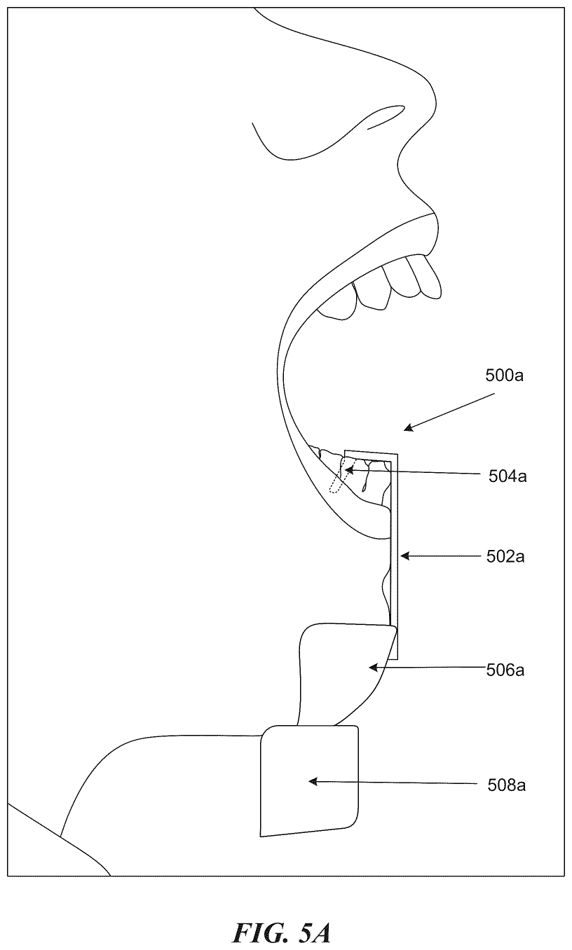

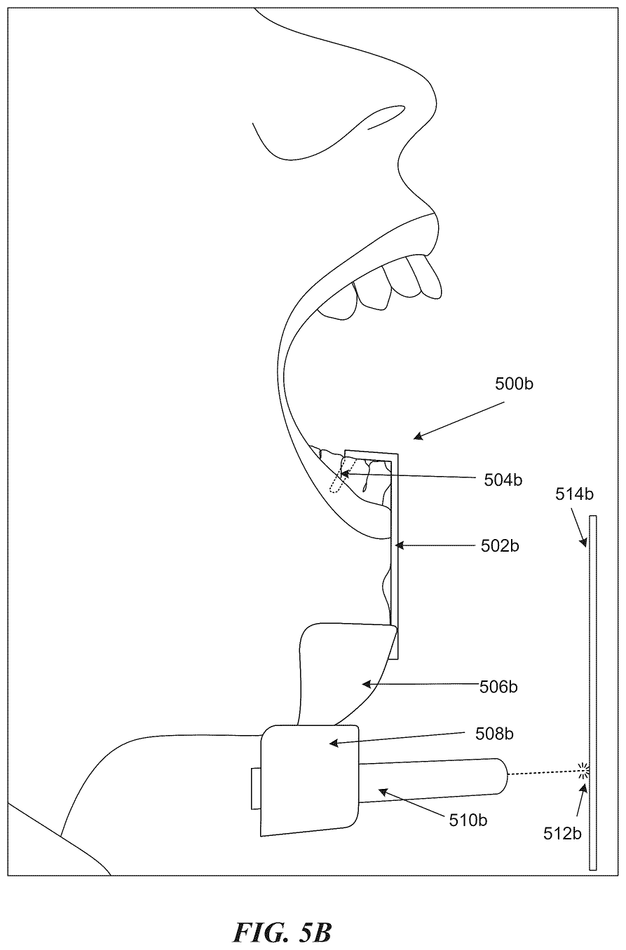

FIG. 5A is a buccal elevation view of the oral device 500a partially placed within the mouth of a patient, illustrating the positioning of the oral device 500a relative to the patient's teeth. The structures and functions of the chin rest 506a, the stem 502a, the oral clasp 504a, and the housing 508a are as described in preceding paragraphs. In some embodiments, the oral clasp 504a can be supplemented with a mouth guard to provide better grip over the teeth or to enhance the comfort of the patient wearing the oral device 500a. In some embodiments, the oral clasp 504a can be replaced with a mouth guard to provide better grip over the teeth or to enhance the comfort of the patient wearing the oral device 500a. In some embodiments, a medical grade dental wax can be used to secure the oral clasp 504a on the teeth.

FIG. 5B is a buccal elevation view of an embodiment of the oral device 500b with a pointing device 510b coupled with the housing 508b, and with the oral device 500b partially placed within the mouth of a patient, illustrating the positioning of the oral device 500b relative to the patient's teeth. The structures and functions of the chin rest 506b, the stem 502b, the oral clasp 504b, and the housing 508b are as described in preceding paragraphs. In some embodiments, the pointing device 510b can be an optical pointing device. In some embodiments, the optical pointing device can be a laser. In some embodiments, the pointing device 510b can be a writing instrument such as a pen or a pencil. A pointer 512b of the pointing device 510b can be observed in an observation plane in front of the device 500b. In some embodiments, the observation plane can include an observation surface 514b that is positioned at a configurable distance in front of the patient. For a patient not suffering from TMD, the pointer 512b can trace a regular path on the observation plane. For a patient suffering from TMD, the pointer 512b can trace an irregular path on the observation plane based on the extent of the patient's disorder. In some embodiments, for a patient suffering from mild TMD, the path traced by the pointer 512b on the observation plane can exceed a first threshold of a measure of irregularity. In some embodiments, for a patient suffering from severe TMD, the path traced by the pointer 512b on the observation plane can exceed a second threshold of a measure of irregularity. In some embodiments, the measure of irregularity can be an angle made by the path traced by the pointer 512b on the observation plane with reference to a vertical plane passing perpendicularly through the observation plane. In some embodiments, the measure of irregularity can be a width of an aggregate path traced by the pointer 512b on the observation plane after a plurality of motions of the TMJ by the patient. In some embodiments, the observation surface 514b can include reference markings to calculate a measure of irregularity of the path traced by the pointer 512b on the observation surface 514b. In some embodiments, the observation plane can include a recording or display device configured to record or persistently represent the path traced by the pointer 512b on the observation plane. In some embodiments, the observation plane can include a photosensitive receptor array configured to electronically record the path traced by the pointer 512b on the observation plane.

In addition to diagnosing TMD in the patient, the device 500b can be used to treat and correct TMD. The patient can exercise her TMJ and train it to trace a regular path on the observation plane. For this, the patient can conduct a plurality of motions of her TMJ while wearing the device 500b, each time observing the path traced by the pointer 512b in the observation plane and moving her jaw so as to reduce the degree of irregularity of the path traced by the pointer 512b in the observation plane. In some embodiments, the observation surface 514b can include reference markings to guide the patient in reducing the degree of irregularity of the path traced by the pointer 512b on the observation surface 514b.

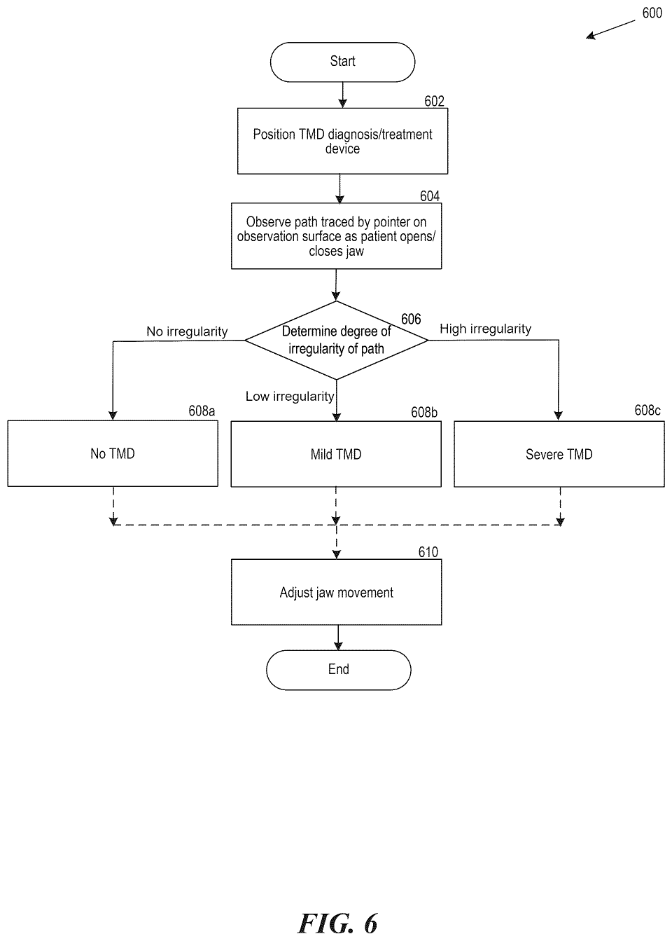

FIG. 6 is a flowchart of a method 600 for diagnosing and treating TMD in the patient using a TMD diagnosis and treatment device. In some embodiments, the device can include a chin rest comprising a superior end and an inferior end, and a stem coupled to the superior end of the chin rest. The stem can comprise a top end and a bottom end, and the bottom end of the stem can be coupled to the superior end of the chin rest. The device can further include a housing configured to receive a pointing device. The housing can be coupled to the inferior end of the chin rest. The device can further include an oral clasp configured to be insertable into a mouth of the patient. The oral clasp can be coupled to the top end of the stem. In some embodiments, the device can further include a pointing device configured to project a pointer on an observation plane in front of the patient. In some embodiments, the pointing device can be coupled to the housing. In some embodiments, the chin rest can have a shape of a quarter-sphere. In some embodiments, the oral clasp can be a dental clasp configured to be worn by the patient on at least one tooth of the patient. In some other embodiments, the oral clasp can be a mouth guard configured to be worn by the patient on at least one tooth of the patient. In some embodiments, the pointing device can be a laser. In some other embodiments, the pointing device can be a writing instrument. In some embodiments, the pointing device can be permanently coupled to the housing. In some other embodiments, the pointing device can be detachable from the housing. In some embodiments, the stem can be adjustable along a longitudinal axis of the stem. In some embodiments, the device can be composed of a plastic material. In some embodiments, the plastic material can be a polylactic acid (PLA). In some other embodiments, the plastic material can be acrylonitrile butadiene styrene (ABS).

At 602, the patient can wear the TMD diagnosis/treatment device by positioning the oral clasp to cover at least a portion of a set of lower teeth of the patient and positioning the patient's chin on the chin rest. At 604, a path traced by the pointer on the observation plane can be observed. In some implementations, the observation plane can include an observation surface. In some implementations, the observation surface can include at least one reference marking for determining the at least one measure of irregularity of the path traced by the pointer.

At 606, a degree of irregularity of the path traced by the pointer on the observation plane can be determined. The degree of irregularity of the path traced by the pointer can be determined to be zero if at least one measure of irregularity of the path does not exceed a first threshold. In some implementations, the at least one measure of irregularity of the path traced by the pointer can be an angle made by the path on the observation plane with reference to a vertical plane passing perpendicularly through the observation plane. In some other implementations, the at least one measure of irregularity of the path traced by the pointer can be a width of an aggregate path traced by the pointer on the observation plane after a plurality of motions comprising opening and closing of the jaw by the patient. At 608a, the patient can be diagnosed to not be suffering from TMD when the degree of irregularity of the path traced by the pointer is zero. The degree of irregularity of the path traced by the pointer can be determined to be low if at least one measure of irregularity of the path exceeds the first threshold. At 608b, the patient can be diagnosed to be suffering from mild TMD when the degree of irregularity of the path traced by the pointer is low. The degree of irregularity of the path traced by the pointer can be determined to be high if the at least one measure of irregularity of the path exceeds a second threshold. The second threshold can be greater than the first threshold. At 608c, the patient can be diagnosed to be suffering from severe TMD when the degree of irregularity of the path traced by the pointer is high. In some implementations, at 610, the method can be further adapted for treating TMJ in the patient by adjusting, by the patient, a position or a motion of the patient's jaw to reduce the degree of irregularity of the path traced by the pointer on the observation plane.

I/we claim:

1. A device for diagnosing and treating a temporomandibular joint (TMJ) disorder (TMD) in a patient, the device comprising:
a chin rest comprising a superior end and an inferior end;
a stem coupled to the superior end of the chin rest,
wherein the stem comprises a top end and a bottom end, and
wherein the bottom end of the stem is coupled to the superior end of the chin rest;
a housing configured to receive a pointing device,
wherein the housing is coupled to the inferior end of the chin rest;
an oral clasp configured to be insertable into a mouth of the patient,
wherein the oral clasp is coupled to the top end of the stem; and
a pointing device configured to project a pointer on an observation plane in front of the patient,
wherein the pointing device is coupled to the housing.

2. The device of claim 1,
wherein the chin rest has a shape of a quarter-sphere.

3. The device of claim 1,
wherein the oral clasp is a dental clasp configured to be worn by the patient on at least one tooth of the patient.

4. The device of claim 1,
wherein the oral clasp is a mouth guard configured to be worn by the patient on at least one tooth of the patient.

5. The device of claim 1,
wherein the pointing device is a laser.

6. The device of claim 1,
wherein the pointing device is a writing instrument.

7. The device of claim 1,
wherein the pointing device is permanently coupled to the housing.

8. The device of claim 1,
wherein the pointing device is detachable from the housing.

9. The device of claim 1,
wherein the stem is adjustable along a longitudinal axis of the stem.

10. The device of claim 1,
wherein the device is composed of a plastic material.

11. The device of claim 10,
wherein the plastic material is a polylactic acid (PLA) or acrylonitrile butadiene styrene (ABS).

12. A method for diagnosing or treating temporomandibular joint (TMJ) disorder (TMD) in a patient, the method comprising:
positioning a TMD diagnosis or treatment device worn by the patient, the TMD diagnosis or treatment device comprising an oral clasp, a chin rest, and a housing coupled with a pointing device configured to project a pointer on an observation plane in front of the patient,
wherein the TMD diagnosis or treatment device is worn by the patient such that:
the oral clasp is positioned to cover at least a portion of a set of lower teeth of the patient, and
the patient's chin is positioned on the chin rest;
observing a path traced by the pointer on the observation plane as a jaw of the patient opens or closes;
determining a degree of irregularity of the path traced by the pointer on the observation plane,
wherein the degree of irregularity of the path traced by the pointer is low if at least one measure of irregularity of the path exceeds a first threshold,
wherein the degree of irregularity of the path traced by the pointer is high if the at least one measure of irregularity of the path exceeds a second threshold,
wherein the second threshold is greater than the first threshold, and
wherein the degree of irregularity of the path traced by the pointer is zero if at least one measure of irregularity of the path does not exceed the first threshold; and
adjusting, by the patient, a position or a motion of the patient's jaw to reduce the degree of irregularity of the path traced by the pointer on the observation plane.

13. The method of claim 12, further comprising:
diagnosing the patient to not be suffering from TMD when the degree of irregularity of the path traced by the pointer is zero.

14. The method of claim 12, further comprising:
diagnosing the patient to be suffering from mild TMD when the degree of irregularity of the path traced by the pointer is low.

15. The method of claim 12, further comprising:
diagnosing the patient to be suffering from severe TMD when the degree of irregularity of the path traced by the pointer is high.

16. The method of claim 12 adapted for treating TMD in the patient, the method comprising:
observing a path traced by the pointer on the observation plane as the patient's jaw opens or closes; and
adjusting a position or a motion of the patient's jaw to reduce the degree of irregularity of the path traced by the pointer on the observation plane.

17. The method of claim 12,
wherein the at least one measure of irregularity of the path traced by the pointer is an angle made by the path on the observation plane with reference to a vertical plane passing perpendicularly through the observation plane.

18. The method of claim 12,
wherein the at least one measure of irregularity of the path traced by the pointer is a width of an aggregate path traced by the pointer on the observation plane after a plurality of motions comprising opening and closing of the jaw by the patient.

19. The method of claim 12,
wherein the observation plane includes an observation surface, and
wherein the observation surface includes at least one reference marking for determining the at least one measure of irregularity of the path traced by the pointer.

20. A device for diagnosing and treating a temporomandibular joint (TMJ) disorder (TMD) in a patient, the device comprising:
a chin rest comprising a superior end and an inferior end;
a stem coupled to the superior end of the chin rest,
wherein the stem comprises a top end and a bottom end, and
wherein the bottom end of the stem is coupled to the superior end of the chin rest;
a housing configured to receive a pointing device,
wherein the housing is coupled to the inferior end of the chin rest; and
an oral clasp configured to be insertable into a mouth of the patient,
wherein the oral clasp is coupled to the top end of the stem.
